(19) United States
(12) Patent Application Publication
(10) Pub. No.: US 2007/0021411 A1
(43) Pub. Date: Jan. 25, 2007

(54) Supersaturated benzodiazepine solutions and their delivery

(76) Inventors: James C. Cloyd, Edina, MN (US); Ronald A. Siegel, Minneapolis, MN (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/429,415

(22) Filed: May 5, 2006

Related U.S. Application Data
(60) Provisional application No. 60/679,718, filed on May 11, 2005.
(60) Provisional application No. 60/775,130, filed on Feb. 21, 2006.

(51) Int. Cl. A61K 31/5513; A61K 31/34

(52) U.S. Cl. 514/221; 514/461

(57) ABSTRACT

The invention describes supersaturated solutions of benzodiazepines, such as diazepam, glycofurol and water and their use for intranasal (NS) administration to combat various disorders.

### CROSS REFERENCE TO RELATED APPLICATIONS

The application claims benefit under 35 U.S.C. § 119(e) to U.S. Ser. Nos. 60/679,718, filed May 11, 2005 (Attorney docket number 186622/US), entitled “Supersaturated Benzodiazepine Solutions and Their Delivery” and 60/775,130, filed Feb. 21, 2006 (Attorney docket number 186622/US/2), entitled “Supersaturated Benzodiazepine Solutions and Their Delivery”, the contents of which are incorporated herein by reference in their entirety including Appendices A, B and C appended thereto.

### FIELD OF THE INVENTION

The invention relates to intranasal delivery of benzodiazepines, such as diazepam, by producing super-saturated drug solutions at point-of-administration. The present invention provides benzodiazepine solutions that are sufficiently stable to prevent the active ingredient from precipitating during the time required for delivery across the nasal mucosal membrane.

### BACKGROUND OF THE INVENTION

The administration by injection (intravenous) of biologically active substances is normally regarded as an acceptable method of administration to achieve a rapid and strong systemic effect and when the active substance is not absorbed or is inactivated in the gastrointestinal tract or by first-pass hepatic metabolism. However, administration by injection presents a range of disadvantages that include the requirement of sterile syringes, skilled personnel, pain and irritation, particularly in the case of repeated injections, extravasation, bleeding, and the risk of infection.

Moreover, intravenous administration of drugs in emergency situations may require trained professionals who are not always available at the time of need. For such drugs, alternative routes of administration are preferred. For example, rectal diazepam has been developed to treat epileptic seizure emergencies in young children. The rectal route promotes rapid absorption, and is theoretically well suited for diazepam administration. This route of administration is generally not acceptable for school-age children and adolescents, however, due to the unwillingness of patients, teachers, school nurses, etc., to administer a drug by this route.

A large number of biologically active substances, including benzodiazepines, have a limited degree of water-solubility. It is often not possible to dissolve a therapeutically effective amount of the biologically active substance in a relatively small volume that can be administered via injection or other means, such as intranasal administration.

For liquid compositions that are to be administered intranasally, it is important that a therapeutically effective amount of the biologically active substance(s) can be dissolved in a volume of less than about 300 μl. Larger volumes are not tolerated well by the individual and the solution will eventually drain out anteriorly through the nostrils or posteriorly toward the pharynx. Thus a portion of the biologically active substance is lost from the absorption site, making it difficult to administer effective dosages. The intranasal volume for human adults is from about 1 μl to about 1000 μl and more preferably from about 50 μl to about 150 μl per nostril.

Children and adults with epilepsy, particularly those who are developmentally disabled, are prone to medical emergencies such as seizure clusters and status epilepticus (SE) that have a devastating effect on health and quality of life. SE and related conditions are among the most common neurological emergencies. An estimated 150,000 patients per year have SE with 42,000 associated deaths. Although relatively common in patients with refractory epilepsy, the overall prevalence of seizure clusters is rare in the epilepsy population. Thousands more suffer from prolonged seizures, which are in themselves serious conditions that can evolve into SE. Children who have SE early in the course of epilepsy are likely to have repeated episodes. Patients with these types of seizure emergencies would benefit from the availability of a safe, effective, easily administered treatment for seizure emergencies. Such a treatment would be an important advance in the management of epilepsy.

Intravenous administration of benzodiazepines (BZDs), including diazepam (DZP), lorazepam (LZP), and midazolam (MDZ), is the most effective and most widely used treatment for seizure emergencies. When given within 30 minutes of the onset of SE, intravenous LZP is effective in 60-80% of patients. Intravenous administration, however, requires skilled personnel and transport to a medical facility. These factors delay initiation of therapy by an average of 85 minutes. Therapy can be further delayed due to difficulty in obtaining intravenous access. A recent study by Alldredge et al. (NEJM 2001) involving patients with SE reported that highly trained paramedics were unable to gain intravenous access 10% of the time. Treatment delay is associated with longer seizure duration, greater difficulty in terminating the seizure, prolonged hospitalization, greater mortality, and reduced quality of life. Out-of-hospital administration of BZDs by parents or other caregivers permits earlier initiation of therapy. However, most routes of administration other than intravenous have shortcomings that diminish their usefulness in treating seizure emergencies. These alternative routes include intramuscular (IM), buccal/sublingual and rectal administration. The usefulness of these routes is largely determined by certain physical-chemical and pharmacokinetic characteristics of BZDs, which are summarized in Table 1.

TABLE 1
Physical-Chemical and Pharmacokinetic
Characteristics of Benzodiazepines

| | Lipid-solubility | | Elimination T½ |
|---|---|---|---|
| Parameter | (Octanol/Water Ratio) | First-pass Effect | (hours) |
| DZP | 309 | Low | 24-48 |
| LZP | 73 | Low | 8-25 |
| MDZ | at pH = 3:34 | High | 0.5-4 |
| | at pH = 7.5:475 | | |

Parents and other caregivers can be trained to give IM injections, but the training is time consuming and, in a medical emergency, the risk of improper injection is high. DZP and LZP absorption following an IM injection is too slow and/or erratic to justify the use of this route of administration. In contrast, IM MDZ is rapidly absorbed, reaching peak serum concentrations within 20-30 minutes and producing a pharmacological effect comparable to IV diazepam within 15 minutes of injection. However, MDZ has not been approved to treat seizures, nor has the ability of non-medical personnel to safely administer an IM injection during a seizure emergency been established. There are no controlled trials evaluating the safety and efficacy of IM MDZ for out-of-hospital treatment of seizure emergencies. Furthermore, the duration of effect following MDZ, regardless of the route of administration, may be quite short given its rapid elimination half-life which ranges from 0.5 to 2.0 hours in children taking enzyme-inducing medications. The need for training, the risks associated with administering an injection, and the undesirable pharmacokinetics of most BZDs following IM injection limit the use of this route for out-of-hospital treatment of seizure emergencies.

Both the buccal (inner pouch of cheek) and sublingual (under the tongue) routes have been proposed as useful alternate methods to administer BZDs. Drug administration by these routes may be difficult in patients who are actively seizuring. Moreover, the solution may be inadvertently swallowed resulting in delayed or reduced absorption due to first-pass metabolism. There is a risk of pneumonitis secondary to aspiration of organic solvents (such as propylene glycol and ethanol) which are present in DZP and LZP liquid formulations. Furthermore, the absorption of LZP tablets taken sublingually is slow due to the time needed to dissolve the tablet and its lower lipid-solubility. There are no reports on the rate or extent of buccal absorption with LZP or DZP liquid formulations. In a study involving 8 healthy volunteers receiving 5 mg of the commercial parenteral MDZ buccally, buccal MDZ reached peak serum concentrations 15-90 minutes after administration with an average bioavailability of 75%, although there was marked inter-subject variability. It is believed that the variability in absorption is due to a first-pass effect that occurs when MDZ solution is swallowed, as is likely to occur to some degree even in conscious, cooperative volunteers. One uncontrolled clinical trial suggests that buccal MDZ is comparable in safety and efficacy to rectal diazepam. Finally, buccal or sublingual administration of drugs to treat seizures is counter-intuitive; families as well as medical personnel are taught not to place anything in the mouth during a seizure. A therapy that requires placement of a drug delivery device or hand into the mouth may be viewed as a weakening of seizure first aid guidelines and may increase the risk of injury to both the patient and the caregiver. The clinical value of buccal MDZ appears limited due to difficulties with buccal administration in actively seizuring patients, its widely variable bioavailability, and the relatively short duration of effect.

Rectal administration is a useful alternative to intravenous injection and can be administered by either medical personnel or primary caregivers. The rate and extent of absorption following rectal administration of BZDs varies according to their physical-chemical and pharmacokinetic properties. As shown in Table 1, LZP is 1/5 as lipid-soluble as DZP and MDZ. Therefore LZP absorption in the rectal cavity, which has a small absorptive surface area, is slow relative to oral administration with peak plasma concentrations occurring 75 minutes after administration. The bioavailability of rectal MDZ is poor, averaging 15-30% of a dose, and widely variable due to poor lipid-solubility at low pH and a high first-pass effect. In contract, rectal DZP which has been extensively studied, produces peak concentrations within 5-10 minutes in children and 15-45 minutes in adults. In controlled clinical trials, rectal DZP has proven highly effective and safe in treating seizure emergencies. Although rectal DZP is safe and effective, reduces medical costs, and improves quality of life, many patients, caregivers, and clinicians are reluctant to consider this mode of therapy during a life threatening seizure—especially in public places—because of personal concerns.

Therefore, a need exists to overcome one or more of the aforementioned disadvantages for the delivery of BZDs. A need exists for a delivery method and/or composition that provides a therapeutically effective amount (concentration) of a benzodiazepine in a minimal volume of a well tolerated liquid which delivers within a few minutes a dose sufficient to prevent or abort a seizure.

### BRIEF SUMMARY OF THE INVENTION

The present invention provides a supersaturated solution of a benzodiazepine dissolved in water and glycofurol. In one aspect, the benzodiazepine is diazepam. In another aspect, the composition provides at least about 10 mg/ml of the benzodiazepine, and in particular, the composition can provide at least between about 5 mg/ml and about about 60 mg/ml of the benzodiazepine, i.e., diazepam.

In one embodiment, the glycofurol percentage of the water and glycofurol combination is between about 40 percent and about 65 percent.

In another embodiment, the glycofurol percentage of the water and glycofurol combination is between about 45 percent and about 60 percent.

In still another embodiment, the concentration of the benzodiazepine is between about 20 mg/ml and about 50 mg/ml.

In yet another embodiment, the concentration of the benzodiazepine is between about 30 mg/ml and about 45 mg/ml, e.g., 40 mg/ml.

In another embodiment, the present invention provides methods for the intranasal administration of a benzodiazepine by providing a therapeutically effective amount of a supersaturated benzodiazepine solution as provided herein. Suitable methods for intranasal delivery include, by spray or by drops. In one aspect, the spray can be created via chaotic advection or turbulent mixing in a suitable delivery chamber.

In another embodiment, the present invention provides methods to induce an improved pharmacologic response in a mammal by nasal administration of a composition comprising a therapeutically effective amount of a supersaturated benzodiazepine solution as provided herein.

In yet another embodiment, the present invention also provides methods to sedate a mammal by the nasal administration of a composition comprising a therapeutically effective amount of a supersaturated benzodiazepine solution as provided herein.

In still yet another embodiment, the present invention provides methods to treat epilepsy by the nasal administration of a composition comprising a therapeutically effective amount of a supersaturated solution of a benzodiazepine as provided herein.

The present invention provides one or more of the following advantages over current technology: it allows for delivery of therapeutically relevant doses in volumes appropriate for nasal administration; the use of the intranasal supersaturated solutions of the invention results in faster absorption which is important when treating seizure emergencies; lower GF content results in less tissue injury when administered intranasally and/or use of DZP in 100% GF in one chamber of a spray bottle or mixing device, such as a microfluidic mixing chamber permits very long storage in container before its used.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 shows equilibrium solubility of diazepam in GF/water cosolvent system at 25° C., 32° C., and 37° C.

FIG. 2 depicts plots of diazepam concentrations in solution versus time in (a) 45% GF, (b) 50% GF, (c) 55% GF, (d) 60% GF cosolvent systems at 25° C., 32° C., and 37° C.

FIG. 3 provides stability data of 40 mg/ml supersaturated diazepam solutions of varying % GF content at 25° C., 32° C., and 37° C.

FIG. 4 provides cumulative permeation of DZP through silicone membranes as a function of time, with upstream vehicle consisting of GF/water cosolvents of 30(⋄), 40(x), and 50(●) vol % GF composition, with DZP in the saturated (S=1) and supersaturated (S=3) states.

FIG. 5a demonstrates cumulative amount of permeated DZP through silicone membranes from 50/50 GF/water vehicle at degrees of saturation S=0.5 (♦), 1 (□), 2 (▴), 3 (◯), 4 (▪), 5 (Δ), and 6.25 (●), as a function of time (mean ±SD, n=3). Lines indicate linear fits through all points except first time point, and when extrapolated to time axis determine time lag, t_{L}.

FIG. 5b depicts steady state flux as a function of S.

FIG. 6 depicts a schematic for the preparation of supersaturated DZP solutions.

FIG. 7 provides a view of herringbone patterns on the floor of a microfluidic mixer.

FIG. 8 provides a visual display of chaotic mixing within a microfluidic mixer, providing a supersaturated solution of DZP in glycofurol and water.

FIG. 9 provides DZP and MDZ concentration-time profiles following intranasal administration.

FIG. 10 illustrates tolerability scores following intranasal Administration (n=2) of DZP or MDZ.

FIG. 11 illustrates comparison of nasal tolerability of 3 candidate formulations of aqueous glycofurol solutions in 12 healthy volunteers.

FIG. 12 provides individual plasma concentrations of supersaturated solutions of diazepam (DZP) in four healthy volunteers.

FIG. 13 provides individual mean plasma concentrations of supersaturated solutions of intranasal diazepam (DZP) and MDZ in four healthy volunteers.

FIG. 14 provides mean global tolerability scores (0-10) of IN DZP.

FIG. 15 is a comparison of intranasal diazepam (5 mg) versus rectal diazepam (5 mg dose adjusted).

FIG. 16 provides composition-dependent viability of MDCK cells treated with GF for 30 min, 1 hr and 2 hrs.

FIG. 17 provides TEER values of MDCK cell monolayer treated with 0% (♦), 10% (□), 20% (▴), 30% (x), 40% (▪), 50% (Δ) glycofurol with assay buffer.

FIG. 18a illustrates percentage of mass transported of [¹⁴C]-mannitol from various GF/AB solutions, 0% (♦), 10% (□), 20% (Δ), 30% (x), 40% (▪), 50% (Δ) glycofurol with assay buffer across MDCK cell monolayer.

FIG. 18b provides permeability of mannitol vs. GF content (mean±S.D., n=6).

FIG. 19a illustrates percentage of mass transported of [¹⁴C]-mannitol from solutions with various DZP concentrations, 0 mg/ml (♦), 1.1 mg/ml (□), 2.2 mg/ml (▴), 3.3 mg/ml (x), 4.4 mg/ml (▪), 5.5 mg/ml (Δ), 6.6 mg/ml (●) in 30/70 GF/AB across MDCK cell monolayer.

FIG. 19b provides permeability of mannitol vs. DZP concentration (mean±S.D., n=6).

FIG. 20a illustrates percentage of mass transported of [¹⁴C]-DZP from various GF/AB solutions, 0% (♦), 10% (□), 20% (▴), 30% (x), 40% (▪), 50% (Δ) glycofurol with assay buffer across MDCK cell monolayer.

FIG. 20b provides transference of DZP vs. GF content (mean±S.D., n=6).

FIG. 21a illustrates percentage of mass transported of [¹⁴C]-DZP from solutions with various DZP concentrations, 0 mg/ml (♦), 1.1 mg/ml (□), 2.2 mg/ml (▴), 3.3 mg/ml (x), 4.4 mg/ml (▪), 5.5 mg/ml (Δ), 6.6 mg/ml (●) in 30/70 GF/AB across MDCK cell monolayer.

FIG. 21b provides permeability of DZP vs. DZP concentration (mean±S.D., n=6).

FIG. 22a demonstrates the cumulative amount of permeated DZP through MDCK cell monolayers from 50/50 GF/water vehicle at degrees of saturation S=1 (♦), 1 (□), 3 (▴), 4 (x), 5 (▪) and 6 (Δ), as a function of time. Lines indicate linear fits through all points except first time point, and when extrapolated to time axis determine time lag, t_{L}.

FIG. 22b is a representation of steady state flux as a function of S. (mean±S.D., n=6).

FIG. 23a is a representation of cumulative amount of permeated DZP through blank filter from 50/50 GF/water vehicle at degrees of saturation S=1 (♦), 1 (□), 3 (▴), 4 (x), 5 (▪) and 6 (Δ), as a function of time (mean±S.D., n=3). Lines indicate linear fits through all points except first time point, and when extrapolated to time axis determine time lag, t_{L}.

FIG. 23b illustrates steady state flux as a function of S.

### DETAILED DESCRIPTION

It has been unexpectedly discovered that supersaturated solutions of benzodiazepines can be prepared that are stable for a sufficient period of time such that the supersaturated solution containing a therapeutic dose can be delivered to an individual in need thereof via intranasal administration. Supersaturated solutions of benzodiazepines provide concentrations of the biologically active substance that are not achieved in non-supersaturated solutions. Use of these unique supersaturated solutions provide the advantage that more of the biologically substance can be delivered in a minimal concentration of delivery vehicle and the supersaturated conditions result in more rapid absorption of the drug. Therefore, increased amounts of the biologically active substance is delivered to the individual with less irritation to the nasal cavity then by conventional drops, sprays or aerosols that only utilize more concentrated organic solvent systems.

In one aspect, the present invention provides a supersaturated solution of a benzodiazepine dissolved in water and glycofurol. In one embodiment, the benzodiazepine is diazepam. In another aspect, the composition provides at least about 10 mg/ml of the benzodiazepine, and in particular, the composition can provide at least about 80 mg/ml, i.e., at least about 40 mg/ml. Therefore, a suitable range of diazepam delivered by the compositions of the invention is in the range of between about 5 mg/ml and about 80 mg/ml.

Typically, the intranasal volume delivered to an individual in need of a benzodiazepine is from about 1 μl to about 1000 μl, more particularly, between about 25 μl and about 250 μl, and more particularly between about 50 μl to about 150 μl per nostril.

The term “benzodiazepine” is recognized in the art and is intended to include any of several similar lipophilic amines used as tranquilizers, sedatives, hypnotic agents or muscle relaxants. Benzodiazepines are a class of drugs with hypnotic, anxiolytic, anticonvulsant, amnestic and muscle relaxant properties. Benzodiazepines are often used for short-term relief of severe, disabling anxiety, insomnia, and/or to prevent or abort severe seizures including status epilepticus. They are believed to act on the GABA receptor GABAA, the activation of which dampens higher neuronal activity.

Suitable benzodiazepines include, for example, alprazolam, bromazepam, brotizolam, camazepam, chlordiazepeoxide, clobazam, chlorazepic acid, clonazepam, clotiazepam, cloxazolam, delorazepam, diazepam, estazolam, ethyl loflazepate, fludiazepam, flunitrazepam, flurazepam, flutazolam, halazepam, haloxazolam, ketazolam, loprazolam, lorazepam, lormetazepam, medazepam, midazolam, nimetazepam, nitrazepam, nordiazepam, oxazepam, oxazolam, pinazepam, prazepam, temazepam, tetrazepam, tofisopam, triazolam and combinations thereof.

Any pharmaceutically acceptable form of the benzodiazepine or combinations of benzodiazepines can be utilized in accordance with the present invention. Generally the selected biologically active substance is provided in the chemical form which has previously been found most efficacious for oral or parenteral delivery. Most commonly, this comprises either the free base or a pharmaceutically acceptable salt.

The terms “subject”, “individual” and “mammal” refer to those in need of treatment with a benzodiazepine. Mammals include but are not limited to, for example, cows, dogs, cats, sheep, horses, bovine, and humans.

It should be understood that the term “comprising” (or comprises) includes the more restrictive terms consisting of and consisting essentially of.

The term “glycofurol” (GF) is recognized in the art and is intended to include the material as described in U.S. Pat. No. 5,397,771, the contents of which are incorporated herein by reference. Glycofurol is commercially available from Sigma Alrich, St. Louis, Mo., USA (CAS number 9004-76-6; product number T3396. Glycofurol is also known as tetrahydrofurfuryl alcohol polyethyleneglycol ether or tetraglycol. The compound has the general formula:

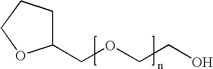

wherein n is 0 to 5.

The term “supersaturated” is recognized in the art and is intended to mean the concentration of a solute that exceeds the intrinsic dissolution capacity of the solution, and which will result over time in the precipitation of a fraction of the solute. For example, DZP is dissolved in GF to which a known amount of water is added. The resultant GF/water solution becomes supersaturated and unstable and some DZP will crystallize out of solution over a period of time.

Alternatively, supersaturated drug solutions can be formed either by evaporation of a volatile solvent component or by mixing a poor solvent into a saturated or subsaturated drug solution, the poor solvent being miscible with the “host” solvent component. Of the two methods, the latter, sometimes called the method of mixed cosolvents, appears to be easier to control and to carry out rapidly and reproducibly.

The stability of supersaturated formulations of the invention can be improved by adding anti-nucleating polymers/crystallization inhibitors, such as methylcellulose, hydroxypropyl methylcellulose and polyvinyl pyrrolidone.

The solutions of the invention can be administered intranasally by providing a therapeutically effective amount of a supersaturated benzodiazepine solution. Suitable methods for intranasal delivery include, by spray or by drops. In one aspect, the spray can be created via chaotic mixing in a suitable delivery chamber.

The biologically active substances of the invention, or compositions thereof, will generally be used in an amount effective to achieve the intended result, for example in an amount effective to treat or prevent the particular disease or condition being treated. The substance(s) may be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder.

For example, administration of a compound to a patient suffering from anxiety provides therapeutic benefit not only when the underlying anxious response is eradicated or ameliorated, but also when the patient reports a decrease in the severity or duration of the symptoms associated with the anxiety following exposure to the stimulus. As another example, therapeutic benefit in the context of anxiety or epilepsy includes an improvement in temporal control following the onset of an epileptic seizure, or a reduction in the frequency or severity of the seizure or the prevention of recurring seizures. Therapeutic benefit also includes halting or slowing the progression of the disease, regardless of whether improvement is realized.

For prophylactic administration, the substance(s) may be administered to a patient at risk of developing one of the previously described diseases or conditions.

Alternatively, prophylactic administration may be applied to avoid the onset of symptoms in a patient diagnosed with the underlying disorder. For example, a compound may be administered to an individual with epilepsy at the onset of an aura or other signal to prevent a seizure. Compounds may also be administered prophylactically to healthy individuals who are repeatedly exposed to stresses known to one of the above-described maladies to prevent the onset of the disorder. For example, a compound may be administered to a healthy individual who is prone to depression or in an effort to prevent the individual from falling into a state of depression or becoming anxious.

The amount of compound administered will depend upon a variety of factors, including, for example, the particular indication being treated, the mode of administration, whether the desired benefit is prophylactic or therapeutic, the severity of the indication being treated and the age and weight of the patient, and the rate and extend of absorption of the particular active compound, etc. Determination of an effective dosage is well within the capabilities of those skilled in the art. Exemplary data is included in the Figures.

For example, an initial dosage for use in animals that achieves a blood or serum concentration of the active compound that is at or above the EC50 of the particular compound as measured by in vitro assay, such as the in vitro assessment of effect on action potentials using patch-clamp procedures in isolated neurons. (White H S et al, General Principles-Discovery and Preclinical Development of Antiepileptic Drugs, In Antiepileptic Drugs, 5th ed, Rene H Levy PhD, Richard H Mattson M D, Brian S Meldrum PhD, Emilio Perucca M D, PhD, eds; Lippincott Williams & Wilkins, 2002, pp 36-48.) Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamonon Press, and the references cited therein.

Initial dosages can also be estimated from in vivo data, such as animal models. Animal models useful for testing the efficacy of compounds to treat or prevent the various diseases described above are well-known in the art. Suitable animal models used to assess antiepileptic activity and to perform dose/concentration ranging studies include maximal electroshock seizure and subcantaneous pentylenetetrazole tests. These are described in White H S et al, General Principles-Discovery and Preclinical Development of Antiepileptic Drugs, In Antiepileptic Drugs, 5th ed, Rene H Levy PhD, Richard H Mattson M D, Brian S Meldrum PhD, Emilio Perucca M D, PhD, eds; Lippincott Williams & Wilkins, 2002, pp 36-48, the contents of which are incorporated herein by reference in their entirety.

The present invention also provides methods to induce an improved pharmacologic response in a mammal by nasal administration of a composition comprising a therapeutically effective amount of a supersaturated benzodiazepine solution as provided herein.

An improved pharmacologic response is one that shows an increase in efficacy over that currently known in the art. In the present invention, the improvement is in the ability to provide increased amounts of benzodiazepines at a more rapid rate by more socially acceptable route of administration to the subject due to the nature of the supersaturated solutions.

In yet another embodiment, the present invention also provides methods to sedate a mammal by the nasal administration of a composition comprising a therapeutically effective amount of a supersaturated benzodiazepine solution as provided herein.

In still yet another embodiment, the present invention provides methods to treat epilepsy by the nasal administration of a composition comprising a therapeutically effective amount of a supersaturated solution of a benzodiazepine as provided herein.

Diazepam (DZP) is a well-accepted drug for the treatment of epileptic seizures. It is presently administered either intravenously or rectally in emergency situations; however, neither of these delivery routes is desirable. Since the nose is one of the most permeable and highly vascularized sites for drug administration, which facilitates rapid absorption and onset of therapeutic action. When diazepam, a highly lipid soluble drug is administered in a supersaturated solution, nasal administration of this drug is a potential alternative to intravenous injections and rectal administration in treatment of seizure emergencies such as acute repetitive seizures, prolonged seizures, or status epilepticus.

Diazepam has low water solubility (0.05 mg/ml), so nasal administration of therapeutic doses in volumes appropriate for the nasal cavity is not feasible. However, in other solvents in which diazepam has higher solubility, the activity of diazepam, and hence its tendency to cross nasal mucosa, is not enhanced. Supersaturation of a benzodiazepine, such as DZP, results in an increased thermodynamic activity of drug substance in the vehicle compared with subsaturated or saturated solutions; hence, a correspondingly higher flux is achieved.

It was found that supersaturated diazepam solutions can be produced by rapid mixing of a diazepam solution in glycofurol (GF), a good solvent for diazepam, with water. Rapid mixing is achieved, for example, by flowing fluids together into a staggered herringbone microfluidic chaotic advection mixer, fabricated by silicon micromachining and micromolding techniques.

A major difficulty presented by the nasal route is the small size of the nasal cavity, such that total dosing volume (which may involve administration through both nostrils) should not generally exceed 150 μl per nostril. Assuming that a DZP dose of 10 mg would be required in adults, a highly concentrated diazepam solution is required. Since DZP's solubility in water (˜0.05 mg/ml) is very low and the dosage requirement is equal to or greater than 0.2 mg/kg, it is difficult to formulate a 100% aqueous solution of DZP for nasal use.

In contrast, the solubility of DZP is much greater in various glycols. In particular, GF is capable of solubilizing 101 mg of DZP per ml. This solubility is sufficient to formulate a highly concentrated DZP solution of 40 mg/ml capable of delivering a therapeutic IN dose in a seizure emergency. Although GF permits a highly concentrated DZP solution, the increased solubility does not guarantee enhanced delivery rate. In fact, a greater solubility increases the drug's affinity for the vehicle, which means the drug is less likely to enter the mucosal membrane—the first step in permeation. Furthermore, high GF concentrations in a formulation increase the risk of local tissue irritation and damage including nose bleeds.

For this reason, supersaturated DZP solutions formulated in GF/water cosolvent mixtures were ascertained for intranasal delivery of DZP as described by the present invention. In supersaturated formulations, the thermodynamic activity of drug in the vehicle is increased relative to its activity in subsaturated or saturated solutions. Hence, an enhanced flux can be expected. Moreover, irritation to the nasal mucosa is substantially lower in mixed GF/water cosolvent systems due to the addition of water. The end result is a faster rate of absorption, which is a desirable characteristic when treating a seizure emergency, with reduced tissue irritation.

At steady state, cumulative permeation across a membrane from a vehicle containing drug at constant concentration to a sink reservoir is normally represented by:
Q=PC*(t−t_{L}) (1)

where C* is the upstream drug concentration, P is the membrane's permeability to the drug, defined as P=KD/h, and t_{L} is the corresponding time lag parameter, t_{L}=h²/6D, with K, D, and h representing respectively the partition coefficient of the drug between upstream vehicle and membrane, the drug's diffusivity in the membrane, and the thickness of the membrane. Recognizing that activity, not concentration, is the true thermodynamic driving force for permeation, and identifying unit drug activity with the solubility in the upstream vehicle, Theeuwes recast Eq. (1) in the form:
Q=TS(t−t_{L}) (2)

where S=C*/C_{S} the degree of saturation of drug in the vehicle, and T is the “transference” of the membrane, defined by T=PC_{S}=(KD/h)C_{S}. When the membrane is inert to the vehicle, D and h, and hence t_{L} are unaffected by vehicle composition. The product KC_{S} is similarly independent of vehicle composition in inert membranes, since an increase in solubility indicates improved compatibility of the drug with the vehicle, and therefore less inclination for drug to partition into the membrane. It follows that transference will also be unaffected by an inert vehicle, and T, rather than P, is the more fundamental characteristic of the membrane. On the other hand, changes in the membrane due to uptake of vehicle will be registered by changes in T and t_{L}. Thus, if the vehicle has no effect on the membrane, the flux across the membrane is only proportional to the drug's degree of saturation in the upstream vehicle. Therefore, if supersaturated upstream solutions are utilized, such as DZP, higher drug delivery rate is achieved.

In a first embodiment, the present invention pertains to a composition comprising a supersaturated solution of a benzodiazepine, water and glycofurol.

In a second embodiment of the composition of the first embodiment, the benzodiazepine is diazepam.

In a third embodiment of either the first or second embodiments, the concentration of a benzodiazepine such as diazepam is between about 10 mg/ml and about 60 mg/ml.

In a fourth embodiment of any of the first through third embodiments, the concentration of the benzodiazepine is about 40 mg/ml.

In a fifth embodiment of any of the first through fourth embodiments, the benzodiazepine is diazepam.

In sixth embodiment of any of the first through fifth embodiments, the glycofurol has the structure

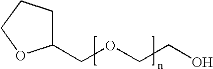

wherein n is 0 to 5.

In a seventh embodiment of any of the first through sixth embodiments, the glycofurol percentage of the water and glycofurol combination is between about 40 percent and about 65 percent.

In an eighth embodiment of any of the first through seventh embodiments, the glycofurol percentage of the water and glycofurol combination is between about 45 percent and about 60 percent.

In a ninth embodiment of any of the first through seventh embodiments, the benzodiazepine concentration is about 40 mg/ml.

In a tenth embodiment of any of the first through eighth embodiments, the benzodiazepine concentration is about 40 mg/ml.

In an eleventh embodiment of any of the first through ninth embodiments, the benzodiazepine is diazepam.

In a twelfth embodiment of any of the first through tenth embodiment, the benzodiazepine is diazepam.

In a thirteenth embodiment, the present invention pertains to a method for intranasal administration of a benzodiazepine, comprising the step of providing a therapeutically effective amount of a supersaturated benzodiazepine solution via intranasal administration, wherein said supersaturated solution comprises a benzodiazepine, water and a glycofurol.

In a fourteenth embodiment of the thirteenth embodiment, the benzodiazepine is diazepam.

In a fifteenth embodiment of either the thirteenth or fourteenth embodiment, the concentration of a benzodiazepine such as diazepam is between about 10 mg/ml and about 60 mg/ml.

In a sixteenth embodiment of any of the thirteenth through fifteenth embodiments, the concentration of the benzodiazepine is about 40 mg/ml.

In a seventeenth embodiment of any of the thirteenth through sixteenth embodiments, the benzodiazepine is diazepam.

In an eighteenth embodiment of any of the thirteenth through seventeenth embodiments, the glycofurol has the structure

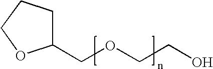

wherein n is 0 to 5.

In a nineteenth embodiment of any of the thirteenth through eighteenth embodiments, the glycofurol percentage of the water and glycofurol combination is between about 40 percent and about 65 percent.

In a twentieth embodiment of any of the thirteenth through nineteenth embodiments, the glycofurol percentage of the water and glycofurol combination is between about 45 percent and about 60 percent.

In a twenty first embodiment of any of the thirteenth through nineteenth embodiments, the benzodiazepine concentration is about 40 mg/ml.

In a twenty second embodiment of any of the thirteenth through twentieth embodiments, the benzodiazepine concentration is about 40 mg/ml.

In a twenty third embodiment of any of the thirteenth through twenty first embodiments, the benzodiazepine is diazepam.

In a twenty fourth embodiment of any of the thirteenth through twenty second embodiment, the benzodiazepine is diazepam.

In a twenty fifth embodiment of any of the thirteenth through twenty fourth embodiments, the supersaturated benzodiazepine solution is administered by spray, i.e., it is delivered intranasally.

In a twenty sixth embodiment of any of the thirteenth through twenty fourth embodiments, the supersaturated benzodiazepine solution is administered by drops, i.e., it is delivered intranasally.

In a twenty seventh embodiment of any of the thirteenth through twenty fifth embodiments, the spray is created via chaotic advection mixing in a microfluidic delivery chamber, or by turbulent mixing.

A twenty eighth embodiment of the invention pertains to a method to sedate a mammal comprising the nasal administration of a composition comprising a therapeutically effective amount of a supersaturated solution of a benzodiazepine, water and a glycofurol.

In a twenty ninth embodiment of the twenty eight embodiment, the benzodiazepine is diazepam.

In a thirtieth embodiment of any of the twenty eighth through twenty ninth embodiments, the concentration of a benzodiazepine such as diazepam is between about 10 mg/ml and about 60 mg/ml.

In a thirty first embodiment of any of the twenty eighth through thirtieth embodiments, the concentration of the benzodiazepine is about 40 mg/ml.

In a thirty second embodiment of any of the twenty eighth through thirty first embodiments, the benzodiazepine is diazepam.

In a thirty third embodiment of any of the twenty eighth through thirty second embodiments, the glycofurol has the structure

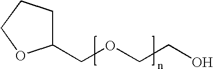

wherein n is 0 to 5.

In a thirty fourth embodiment of any of the twenty eighth through thirty third embodiments, the glycofurol percentage of the water and glycofurol combination is between about 40 percent and about 65 percent.

In a thirty fifth embodiment of any of the twenty eighth through thirty fourth embodiments, the glycofurol percentage of the water and glycofurol combination is between about 45 percent and about 60 percent.

In a thirty sixth embodiment of any of the twenty eighth through thirty fourth embodiments, the benzodiazepine concentration is about 40 mg/ml.

In a thirty seventh embodiment of any of the twenty eighth through thirty fifth embodiments, the benzodiazepine concentration is about 40 mg/ml.

In a thirty eighty embodiment of any of the twenty eighth through thirty sixth embodiments, the benzodiazepine is diazepam.

In a thirty ninth embodiment of any of the twenty eighth through thirty seventh embodiments, the benzodiazepine is diazepam.

In a fortieth embodiment of any of the twenty eighty through thirty ninth embodiments, the supersaturated benzodiazepine solution is administered by spray, i.e., intranasally.

In a forty first embodiment of any of the twenty eighth through thirty ninth embodiments, the supersaturated benzodiazepine solution is administered by drops, i.e., intranasally.

In a forty second embodiment of any of the twenty eighty through fortieth embodiments, the spray is created via chaotic mixing in a microfluidic delivery chamber and is delivered intranasally.

In a forty third embodiment the present invention pertains to a method to treat epilepsy comprising the nasal administration of a composition comprising a therapeutically effective amount of a supersaturated solution of a benzodiazepine, water and a glycofurol.

In a forty fourth embodiment of the forty third embodiment, the benzodiazepine is diazepam.

In a forty fifth embodiment of any of the forty third or forty fourth embodiments, the concentration of a benzodiazepine such as diazepam is between about 10 mg/ml and about 60 mg/ml.

In a forty sixth embodiment of any of the forty third through forty fifth embodiments, the concentration of the benzodiazepine is about 40 mg/ml.

In a forty seventh embodiment of any of the forty third through forty sixth embodiments, the benzodiazepine is diazepam.

In a forth eighty embodiment of any of the forty third through forty seventh embodiments, the glycofurol has the structure

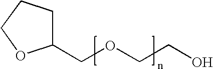

wherein n is 0 to 5.

In a forty ninth embodiment of any of the forty third through forty eighth embodiments, the glycofurol percentage of the water and glycofurol combination is between about 40 percent and about 65 percent.

In a fiftieth embodiment of any of the forty third through forty eighth embodiments, the glycofurol percentage of the water and glycofurol combination is between about 45 percent and about 60 percent.

In a fifty first embodiment of any of the forty third through forty ninth embodiments, the benzodiazepine concentration is about 40 mg/ml.

In a fifty second embodiment of any of the forty third through fiftieth embodiments, the benzodiazepine concentration is about 40 mg/ml.

In a fifty third embodiment of any of the forty third through fifty first embodiments, the benzodiazepine is diazepam.

In a fifty fourth embodiment of any of the forty third through fifty second embodiments, the benzodiazepine is diazepam.

In a fifty fifth embodiment of any of the forty third through fifty fourth embodiments, the supersaturated benzodiazepine solution is administered by spray, i.e., intranasally.

In a fifty sixth embodiment of any of the forty third through fifty fourth embodiments, the supersaturated benzodiazepine solution is administered by drops, i.e., intranasally.

In a fifty seventh embodiment of any of the forth third through fifty fifth embodiments, the spray is created via chaotic advection mixing in a microfluidic delivery chamber, or by turbulent mixing.

Materials and Methods

Materials

Diazepam and glycofurol were purchased from Sigma-Aldrich Chemical Co. (St. Louis, Mo., USA). HPLC grade acetonitrile was purchased from Fisher Scientific (Fair Lawn, N.J., USA). Silicone membranes of thickness 75 μm were generously provided by Dow Corning Corporation (Midland, Mich., USA). All other chemicals were purchased from Mallinckrodt & Baker (Paris, Ky., USA) and were used as received. Premium de-ionized water was used throughout the experiments presented herein.

HPLC Analysis

HPLC analysis of diazepam was performed using a Shimadzu LC-6A pump, a SCL-6A system controller, a SIL-6A autosampler, a SPD-6A UV spectrophotometric detector set at 232 nm and a CR501 integrator. The stationary phase was a YMC™ reverse-phase butyl (C4) S-3 3.0×150 mm column (pore size 120 angstrom). A ChromTech A-318 inline filter was placed between the sample injection valve and the HPLC column. The mobile phase consisting of 60% potassium phosphate buffer (20 mM, pH=6) and 40% acetonitrile was pumped through the column at a rate of 0.4 ml/min. Samples were injected via a 50 μl loop and the retention time was ≈10.2 min. Calibration curves were constructed on the basis of the peak height measurements using standard solutions of known concentrations.

Solubility Studies

Equilibrium solubility of diazepam in GF/water cosolvent mixtures, varying from 5 to 60 vol % GF, was determined at 25° C., 32° C., and 37° C. Saturated solutions were prepared by adding excess drug to the cosolvent mixtures. After shaking for 48 hrs at these temperatures, the resulting suspensions were centrifuged for 15 min at 13000 RPM. The supernatant was then removed, properly diluted, and analyzed using HPLC. Solubility studies were performed in duplicate at each condition.

Preparation of Supersaturated Solutions

Supersaturated DZP solutions were prepared at room temperature by first dissolving an appropriate quantity of DZP in glycofurol, and then adding either pure water or a GF/water cosolvent mixture, dropwise and under constant agitation by a vortex mixer. Occasionally, small transient milky phases appeared. These phases, which probably included nuclei of DZP crystals or amorphous pre-nuclei, formed at the interface between the two liquids, were eliminated by further agitation. All solutions were prepared freshly before use.

Temporal Stability Studies

The temporal stability of 40 mg/ml supersaturated DZP solutions prepared in 45%, 50%, 55%, and 60% glycofurol and water cosolvent mixtures was investigated at 25° C., 32° C., and 37° C. At various time points after preparation, aliquots of these solutions were taken and centrifuged as above. The supernatant was then analyzed using HPLC. Temporal stability studies were carried out in triplicate at each condition.

Diffusion Studies

A water-jacketed, temperature-controlled Franz diffusion cell (PermeGear Inc., Riegelsville, Pa.) was used to investigate permeation of DZP through silicone membranes. The cell had a diffusional surface area of approximately 1 cm² and a receptor volume of approximately 8 ml.

The silicon membrane was cut to the appropriate size and allowed to soak overnight in PBS. Silicone grease was used to produce a leak-proof seal between the membrane and the flanges of the two half-cells.

The donor phase was 0.5 ml of a DZP solution in GF/water cosolvent of specified composition, with DZP either in the saturated or supersaturated state. The donor compartment was occluded to prevent solvent evaporation.

Degassed phosphate buffered saline (PBS, pH 7.4), served as the receptor phase, and magnetic stirring was employed to ensure sink conditions. The sampling arm of the receptor compartment was covered with Parafilm to prevent evaporation, except when samples were drawn. At predetermined intervals 200 μl of the receptor phase was removed and replaced with an equal volume of pre-thermostated and degassed PBS. The samples were assayed by HPLC.

The cumulative amount of permeated DZP through the silicone membrane (mass/area) was plotted as a function of time for each cosolvent-DZP concentration condition. Steady state flux and time lag values were determined by linear regression on these plots, ignoring the first time point, which preceded the establishment of steady state.

All diffusion studies were carried out in triplicate, with the receptor phase at 37° C.

Results

Solubility Studies

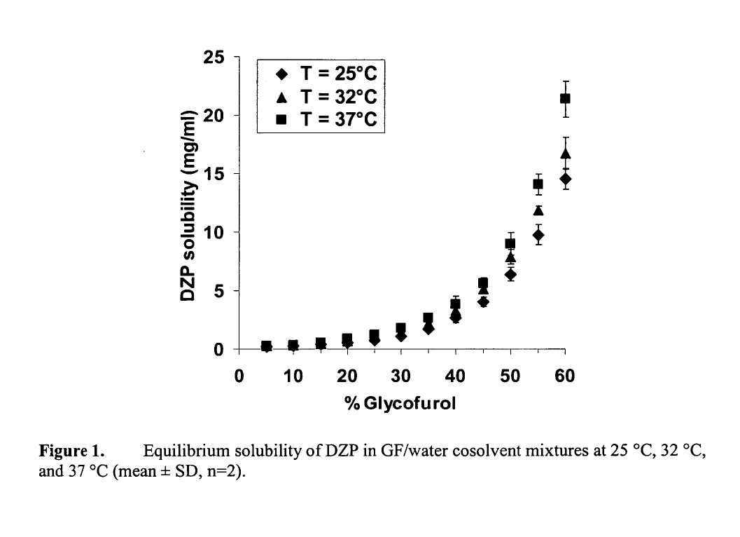

Knowledge of a drug's solubility for each vehicle composition is imperative for preparation of supersaturated formulations. DZP has an exceedingly low solubility in water (C_{S}=0.05 mg/ml), but it is highly soluble in glycofurol (C_{S}=101 mg/ml). FIG. 1 shows the equilibrium solubility of DZP in GF/water cosolvent systems with GF composition varying between 5 and 60 vol %, at 25° C. (room temperature), 32° C. (nasal cavity temperature), and 37° C. (core body temperature). Solubility of DZP increases smoothly and convexly with increasing glycofurol content. Temperature also has a minor but noticeable effect.

Temporal Stability Studies

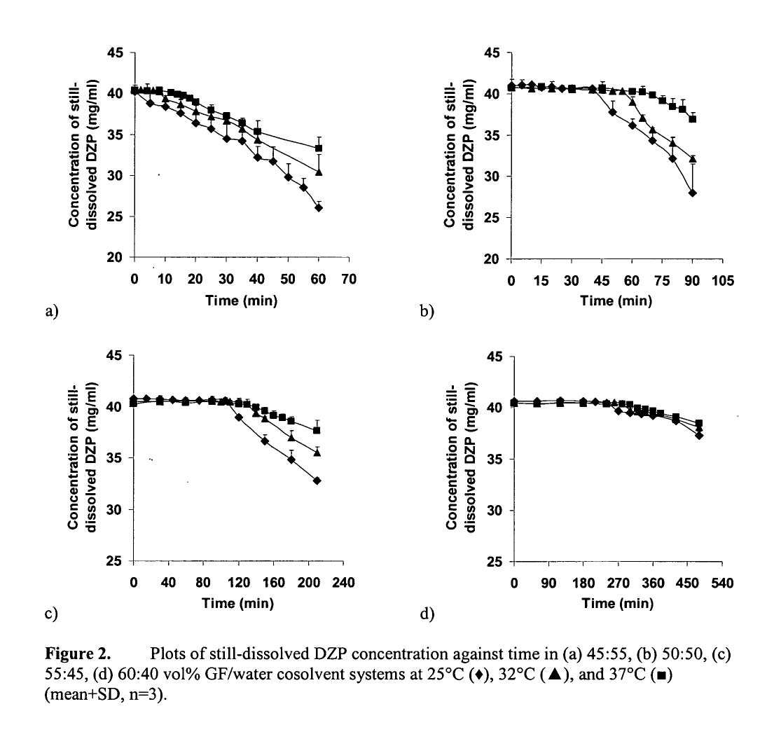

The solubility results presented above represent thermodynamic equilibrium. Since supersaturated DZP solutions were of interest, which are thermodynamically unstable and ultimately may crystallize, the temporal stability of supersaturated 40 mg/ml (the concentration relevant to intranasal delivery, see above) DZP solutions in several GF/water compositions and at 25° C., 32° C., and 37° C. was determined. The concentration of still-dissolved DZP versus time is plotted in FIG. 2, for 45:55, 50:50, 55:45, and 60:40 vol % GF/water vehicles. It was difficult to prepare 40 mg/ml supersaturated DZP solutions in cosolvents with less that 45 vol % GF, whereas cosolvent systems with GF content greater than 60 vol % do not generally meet the tolerability requirement for intranasal administration.

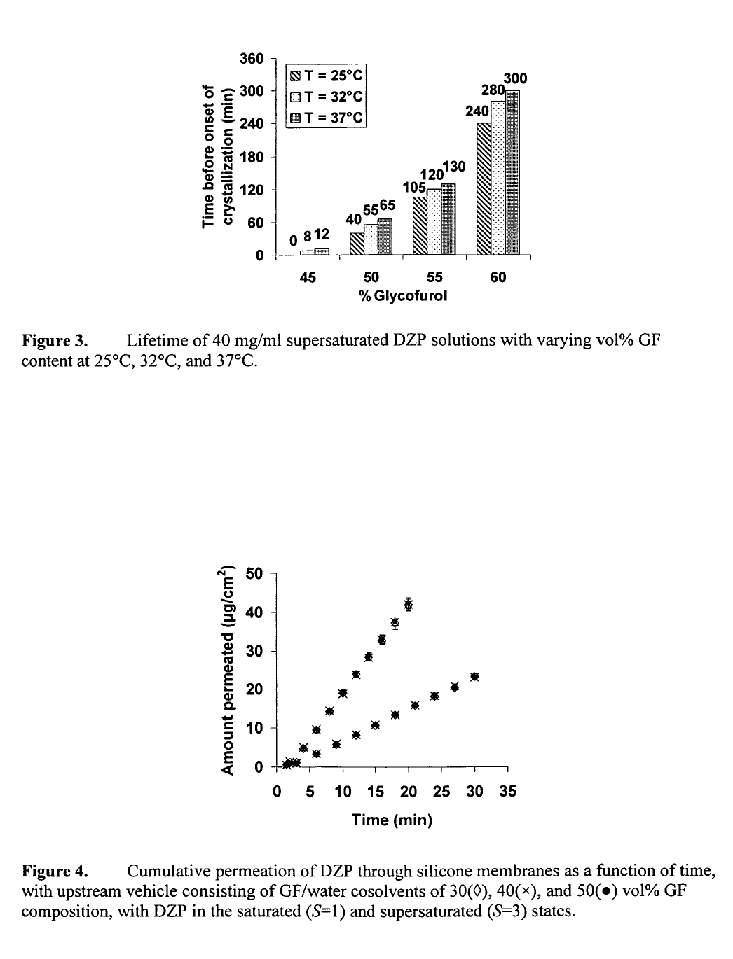

Almost all curves in FIG. 2 feature an initial period during which all DZP remains in solutions. A break occurs at a well defined time point, after which the dissolved DZP concentration decreases and crystals were observed to form. FIG. 3 shows the joint effect of GF/water composition and temperature on the duration before commencement of crystallization with 40 mg/ml supersaturated DZP solutions. The lifetime of these supersaturated solutions increases with increasing GF content and increasing temperature. Both of these factors increase DZP solubility and hence diminish the degree of saturation given a fixed concentration of DZP.

Diffusion Studies

Effect of Vehicle on Permeability and Time Lag

To demonstrate that transference of DZP in the silicone membrane is unaffected by the vehicle, permeation was first measured with upstream solutions of DZP in different GF/water vehicle compositions (30, 40, and 50 vol % GF), with DZP in both the saturated (S=1) and the supersaturated (S=3) states. Time courses of accumulated transport of DZP across the membrane under this matrix of conditions are shown in FIG. 4. For each value of S, transport curves for each vehicle composition are essentially identical, and both sets of curves share an essentially identical time lag.

Calculated values of steady state flux of DZP through the membrane, J_{SS}, transference (T=J_{SS}/S) and time lag are presented in Table 2. As expected from the curves in FIG. 4, neither transference nor time lag is significantly affected by either vehicle composition or drug concentration. These results provide that the observed permeation enhancement through the silicone membranes used was a result supersaturation.

TABLE 2
Solubilities of diazepam (DZP), steady state fluxes, transferences and time
lags measured for DZP crossing silicone membranes (75 μm thickness) from
glycofurol (GF)/water cosolvents into aqueous sinks, with different glycofurol
contents in the donor vehicle (mean ± SD, n = 3).

| | | Solubility at | | | |
|---|---|---|---|---|---|
| | | 25° C. (mg/ml) | Steady State Flux | Transference | Time Lag |
| S | % GF | (n = 2) | [μg/(cm² · min)] | [μg/(cm² · min)] | (min) |
| 1 | 30 | 1.102 ± 0.102 | 0.823 ± 0.030 | 0.823 ± 0.030 | 1.876 ± 0.165 |
| | 40 | 2.702 ± 0.425 | 0.826 ± 0.019 | 0.826 ± 0.019 | 1.793 ± 0.020 |
| | 50 | 6.398 ± 0.573 | 0.824 ± 0.011 | 0.824 ± 0.011 | 1.842 ± 0.119 |
| 3 | 30 | 3.306 ± 0.306 | 2.309 ± 0.070 | 0.770 ± 0.023 | 1.806 ± 0.135 |
| | 40 | 8.106 ± 1.275 | 2.310 ± 0.055 | 0.770 ± 0.018 | 1.787 ± 0.091 |
| | 50 | 19.19 ± 1.719 | 2.321 ± 0.095 | 0.774 ± 0.032 | 1.755 ± 0.134 |

Effect of Supersaturation on DZP Permeation

Having established that the vehicle has no effect on the membrane, permeation of DZP across the silicone membrane was measured from solutions at different degrees of saturation in a 50:50 GF/water vehicle, which was chosen based on the temporal stability of DZP in that vehicle (FIGS. 2b, 3). Permeation experiments were carried out over a period of 20 minutes. The cumulative amounts of permeated DZP from solutions with degrees of saturation S=0.5, 1, 2, 3, 4, 5, and 6.25 are plotted as a function of time in FIG. 5a, and the steady state fluxed calculated from these data are plotted against degree of saturation in FIG. 5b. Steady state flux increased nearly linearly with increasing degree of saturation, demonstrating the ability to enhance transport using supersaturation. It was noted that beyond S=3 the relation between steady state flux and degree of saturation falls below linearity and appears to approach an upper limit. The time lag was negligibly affected by DZP concentration, as shown in Table 3.

TABLE 3
Ratios of fluxes from supersaturated DZP in 50/50 glycofurol/water
solutions at different degrees of saturation to flux from saturated
solution in the same vehicle, and corresponding time lags measured
for 75 μm silicone membranes (mean ± SD, n = 3).

| S | Ratio of Fluxes | Time Lag (min) |
|---|---|---|
| 1 | 1 | 1.842 ± 0.119 |
| 2 | 1.994 ± 0.013 | 1.785 ± 0.279 |
| 3 | 2.874 ± 0.118 | 1.754 ± 0.135 |
| 4 | 3.123 ± 0.165 | 1.768 ± 0.117 |
| 5 | 3.377 ± 0.048 | 1.755 ± 0.164 |
| 6.25 | 3.581 ± 0.179 | 1.738 ± 0.114 |

In the present intranasal diazepam in glycofurol/water formulations, supersaturation plays at least two roles. First, it permits the formulation to increase the water content, and hence lower the content of the irritating organic cosolvent GF, while still permitting an adequate dose of a low water soluble drug to be administered into a very limited space. Second, supersaturation provides an enhanced driving force for permeation across the nasal mucosa, with accelerated absorption. In the treatment of seizure emergencies, rapid onset of response is of interest, partly to relieve immediate symptoms, but also to prevent damage to the CNS which may occur during a seizure.

In the present invention, the solubility properties of DZP in mixed GF/water cosolvent vehicles at various cosolvent compositions was investigated at three relevant temperatures, the temporal stability of freshly prepared supersaturated solutions, and the potential for enhanced transport of DZP across membranes. The present invention provides that DZP solubility is a tunable function of vehicle composition and that it increases somewhat with temperature. The present invention also provides that for GF cosolvent composition at 50 vol % or above, 40 mg/ml solutions are temporally stable for at least 40 min, which is much longer than the time that would be needed for rapid-onset of drug effect, e.g. a time-to-peak plasma concentration below 20 min. Finally, the present invention provides that Eq. (2), relating transmembrane flux to degree of saturation hold for concentration up to 3× solubility, at least for silicone membranes. Beyond this point, increases in supersaturated DZP concentration result in only minor increments in flux.

One purpose in using an in vitro membrane model was to investigate the limits of the use of supersaturation in a system where other means of penetration enhancement, such as alteration of the membrane by the vehicle, could be eliminated as factors. The invariance of lag time under all conditions, and the practical confirmation of Eq. (2) up to S=3 indicate that the observed permeation enhancement is due to the enhanced activity of the permeating solute.

Microfluidic Mixing Device

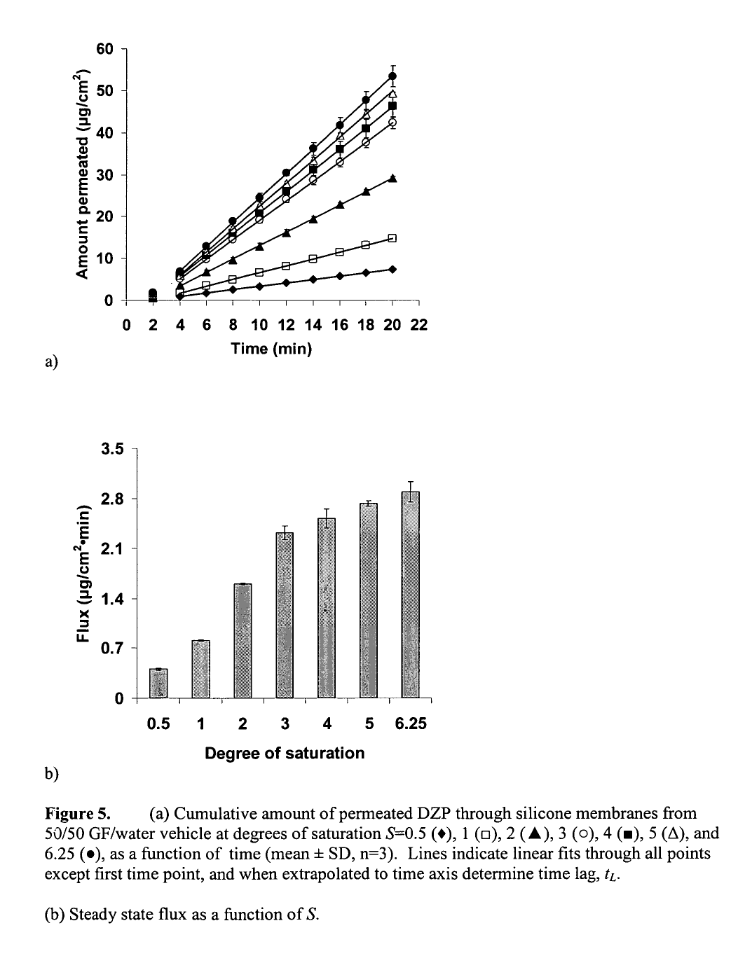

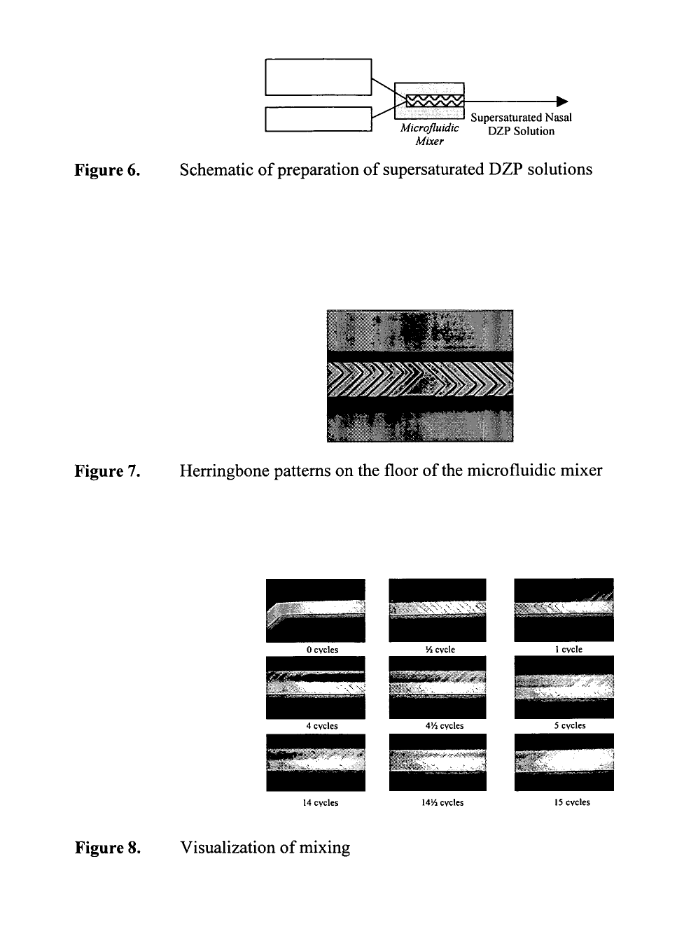

Because of the potential for precipitation of supersaturated DZP solutions, they generally cannot be easily formulated for storage unless an anticrystallization agent as described herein is added. The supersaturated solutions used in the experiments described in the previous subsections were prepared freshly before use by rapidly combining solutions of DZP in GF with water/GF mixtures, followed by rapid vortexing. Because of the small volumes of to be prepared for intranasal administration, this method is not ideal for drug delivery. As an alternative, a simple means for preparing supersaturated DZP solutions is provided: Briefly, a saturated DZP solution in a particular water/GF cosolvent is mixed with water in a microfluidic mixing chamber. A suitable example of a microfluidic mixing chamber is shown in FIG. 6.

Microfluidic mixers (mixing chambers) are known in the art. Examples include those described in U.S. Pat. Nos. 7,011,791, 6,935,772, 6,919,046, 6,907,895, 6,901,963, 6,890,093, 6,877,892, 6,802,489, 6,676,835, 6,568,052, 6,331,073, 6,210,128 and 6,170,981, the contents of which are incorporated herein in their entirety for all purposes.

The microfluidic mixer generally consists of a narrow channel whose inlet makes a “Y”, such that the two fluids to be mixed are fed from two branches into a common stem. The bottom of the channel (FIG. 7) contains micro-ridges in a herringbone configuration, where the “sense” of the herringbone alternates as one moves down the channel. The channel's width and depth are 200 μm and 90 μm, respectively, and the micro-ridges have height and width 15 μm and 50 μm, respectively. As the fluids move together down the channel, the ridges cause them to roll over. Because of the herringbone, the rolls break up and recombine in such a way that the initially distinct fluid layers become interspersed, allowing rapid diffusional mixing to occur between the fluids. As an example, the sequence of images at FIG. 8 records passage of a transparent fluid (top) and a fluorescent fluid, introduced through the Y-inlet, through the channel. The transparent fluid is invisible. However, it is seen that mixing commences as the fluids pass through a few cycles of “sense reversal” of the herringbone ridges. Mixing is nearly complete after 15 cycles, corresponding to a channel length of 2.8 cm from the Y-inlet.

Clinical Studies

Nasal Administration of Diazepam and Midazolam Preparations

Methods: Two healthy volunteers were each given IV DZP, IN DZP, IV MDZ, and IN MDZ. A commercial parenteral formulation (Versed®-5 mg/ml) of MDZ was used for both IV and IN MDZ administration, whereas a parenteral DZP formulation (Diazepam Injectable, USP, 5 mg/ml) was used for IV DZP administration and an oral solution (Diazepam Intensol®-5 mg/ml) was used for IN DZP administration. Serial blood samples were collected over 48 hours and analyzed using HPLC. Analog tolerability scales were administered to the two subjects to determine overall tolerability and level of sedation at various time points after drug administration. Five mg doses (1 ml) of both DZP and MDZ given nasally were drawn up into a syringe. Approximately 0.5 ml (2.5 mg) was dripped into each nostril over a period of 30 seconds.

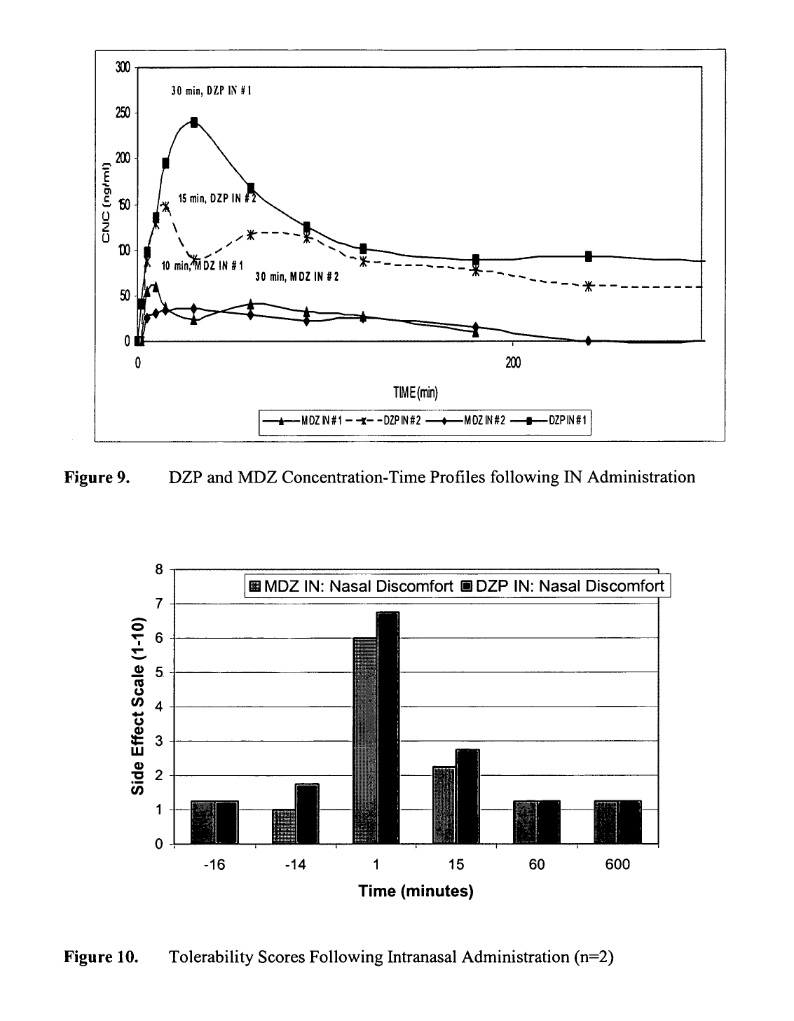

Results: FIG. 9 shows the concentration vs. time profile of IN DZP and MDZ. As presented in Table 4, the rates of absorption as measured by the time to maximum concentration were comparable, but the bioavailability and elimination half-life were greater for DZP. Subject self-rating of nasal discomfort is shown in Table 4 and FIG. 10. Both DZP (solvents are 40% propylene glycol and 10% ethanol) and MDZ (pH=3) caused significant discomfort.

TABLE 4
Comparison of MDZ and DZP Pharmacokinetics and Patient Tolerability
following IN Administration of a 5 mg Dose (n = 2)

| | Diazepam | Midazolam |
|---|---|---|
| Tmax (min) | 22.5 | 20 |
| Half-life (hours) | 29.2 | 1.54 |
| Bioavailability (0-180 minutes) | 92% | 67.5% |
| Nasal Discomfort | 1.5 | 1.0 |
| Nasal Discomfort (maximum) | 6.8 | 6.0 |

Conclusions: Both DZP and MDZ are rapidly absorbed, but DZP exhibits greater and more consistent bioavailability and a longer elimination half-life. Nasal discomfort and the relatively dilute concentration (5 mg/ml) of the commercially available DZP and MDZ formulations limit their use for IN administration.

The Tolerability of Nasally Administered Glycofurol Solutions in Healthy Volunteers

Methods: Each of the candidate solutions of GF (without drug) was evaluated in 12 healthy volunteers using a randomized, three treatment, single-blind design. 3 candidate GF and water formulations: 60% GF, 80% GF and 100% GF with a one-week washout period were administered. A volume of 0.5 ml of each solution was instilled in one nostril by dropper. Normal saline, as an internal control, was applied in a similar manner to the opposite nostril. Subjects were asked to rate tolerability at baseline; immediately after administration; and 5, 15, and 30 minutes after administration. In addition, subjects completed a questionnaire to further characterize the nature of any discomfort they experienced. Approximately 0.5 ml of the appropriate liquid was dripped into each nostril over a period of 30 seconds. GF/Water solutions in one nostril, saline in other nostril.

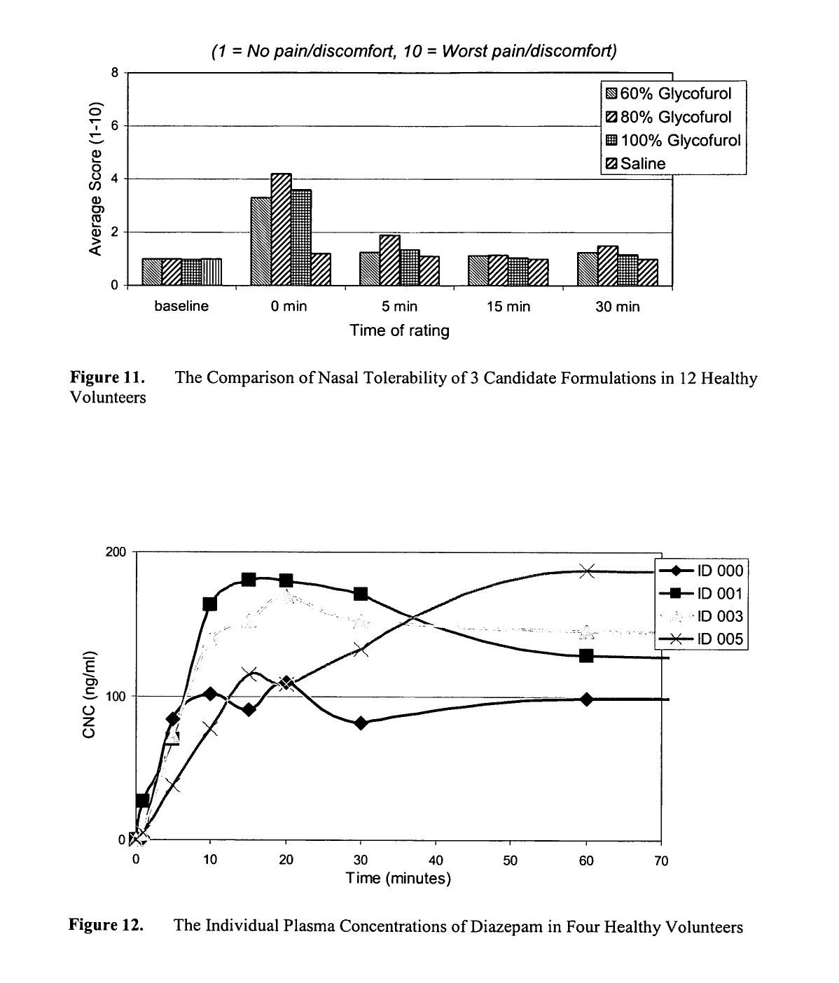

Results: As shown in FIG. 11, the data indicate that the 60% GF formulation demonstrated a modest improvement in tolerability scores. The intolerability was short-lived with improvement to baseline occurring within 5 minutes. The 60% GF and water formulation was chosen for further study in order to determine its diazepam solubility and stability. Data indicated that a lower concentration of GF/water may be used for clinical studies. This may also improve tolerability of the formulation.

Tolerability and Bioavailability Study Comparing Intranasal DZP and MDZ in Healthy Volunteers

Background: Although there are no adequately controlled clinical trials demonstrating its safety and efficacy, IN MDZ has been cited in the literature for use as a preoperative sedative and for treatment of seizures. There are very few reports describing intranasal administration of other benzodiazepines. As a result of these reports, some clinicians prescribe IN MDZ using the parenteral formulation for out-of-hospital treatment of seizure emergencies. The purpose of this study was to determine if there are pharmacokinetic advantages of using either DZP or MDZ for intranasal administration.

Methods: The study consisted of a randomized, single-blind, four-way crossover study to determine tolerability and bioavailability of intranasal DZP and MDZ. The intranasal arms were placebo-controlled using normal saline administered nasally. Four healthy volunteers received each of four treatments, with one treatment assigned randomly each day over the course of four days: 5 mg of the supersaturated DZP solution intranasally, 5 mg IV DZP (Diazepam Injectable, USP), 5 mg IN MDZ of the parenteral MDZ solution (5 mg/ml), and 5 mg IV MDZ. The supersaturated DZP solution was prepared as follows: 80 mg of DZP was added to 0.9 mls of 100% GF mixed by shaking the tube. In a second test tube, 0.3 ml of GF was added to 0.8 ml of water. The contents of the second tube were then added by dropper into tube one while gently the tube. The DZP/GF/Water mixture was then vortexed for 60 seconds. The resulting solution contained 40 mg/ml of DZP in a 60% GF/Water solution. Using a syringe, 0.25 ml of the DZP/GF/Water solution was withdrawn from the test tube. A 0.125 ml dose of DZP was instilled into each nostril over 30 seconds. The total volume was 0.25 ml which delivers 5 mg of DZP. The parenteral MDZ solution was used for the nasal administration with 0.5 ml instilled into each nostril over 30 seconds.

Serial blood samples were collected over 24 hours for MDZ and 48 hours for DZP. Subjects' plasma samples were assayed using HPLC to quantify the concentrations of DZP and MDZ at varying time points. Plasma samples were extracted using a liquid-liquid extraction of sodium hydroxide and methyl-t-butyl-ether. Subjects completed tolerability questionnaires and analog scales to determine tolerability and levels of sedation at various time points after drug administration.

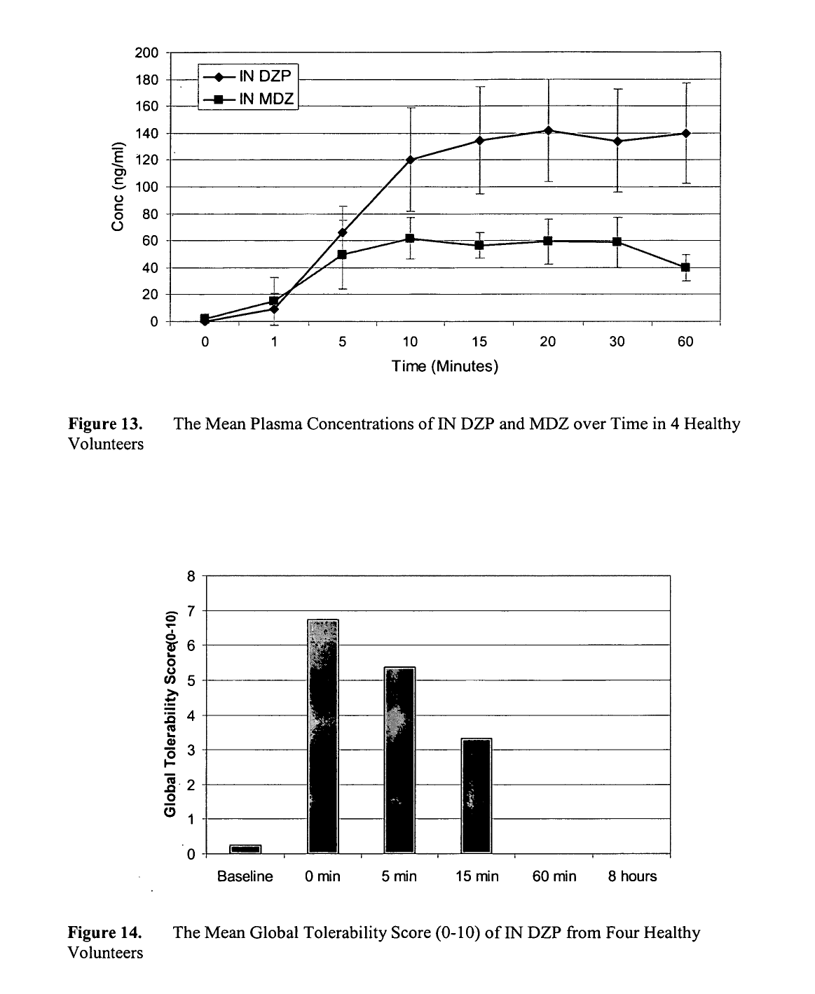

Results: Serial blood samples were collected to determine the subjects' pharmacokinetic profile (FIGS. 12 and 13). One subject had an unusually long time to maximum concentration, which skewed the time to peak concentration. The mean pharmacokinetic parameters are shown in Table 5. The mean global tolerability scores are presented in FIG. 14. Subjects reported moderate intolerability scores with scores returning to baseline within 60 minutes.

TABLE 5
Comparison of Mean Pharmacokinetic Characteristics
between 5 mg Dosed of Intranasal DZP and MDZ in a
60% GF/Water Solution in Four Healthy Volunteers

| | Diazepam | Midazolam |
|---|---|---|
| Cmax (ng/ml) | 28.8 | 13.3 |
| Cmax (ng/ml) | 161.8 | 64.6 |
| Half-life (hours) | 21.5 | 2.2 |
| % F (∞) | 99.4% | 97.9% |

The half-life and bioavailability values are crude estimates. Blood samples were not collected from 1 hour to 8 hours for the midazolam samples and from 1 hour to 24 hours for the diazepam samples. The lack of data between these points precludes a full characterization of the area under the concentration-time curve, half-life, and the fraction of dose that was absorbed.

Conclusion: Both IN DZP and MDZ formulations are extensively and rapidly absorbed. DZP has a longer elimination half-life than MDZ. The tolerability scores are moderately high, but return to baseline values within 60 minutes.

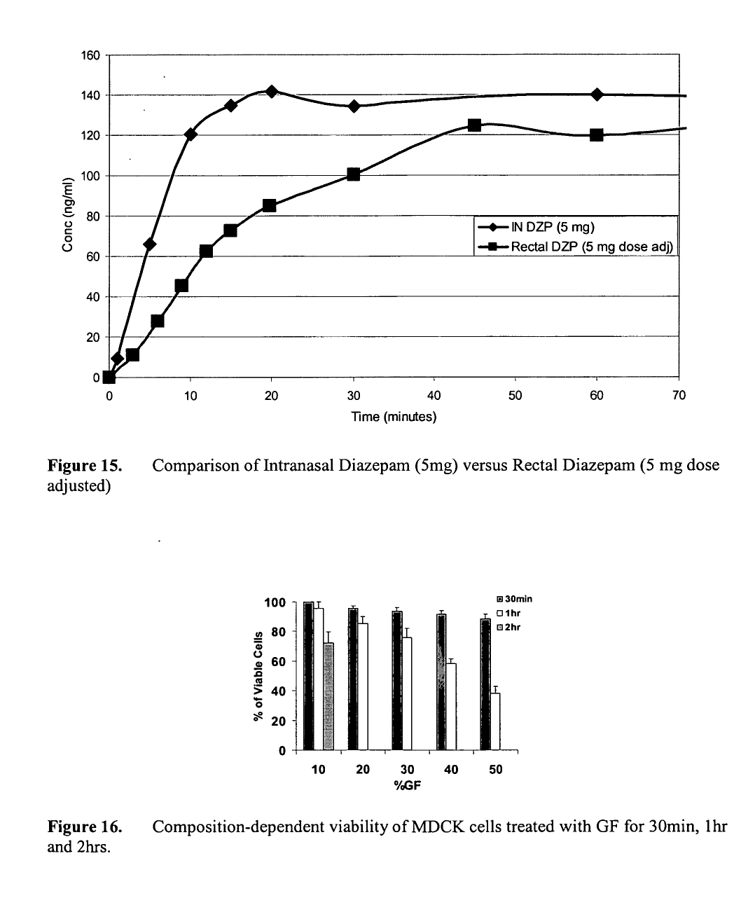

Using published data from studies on rectally-administered DZP, the concentration-time course for PR and IN DZP were compared. IN DZP data obtained from the study described in section above was used as well as data for rectal DZP obtained from the publication Cloyd JC et al, Epilepsia, 1998 Assuming linear pharmacokinetics, expected concentrations were computed following an adjustment from 15 mg to 5 mg of a rectal dose. The resulting concentrations were compared to the concentrations observed from the present invention, intranasal diazepam (5 mg) study (FIG. 15). The purpose was to determine how the preliminary intranasal diazepam data compared to the FDA-approved rectal diazepam. The time to maximum concentration was less with nasal diazepam (28 min.) than rectal diazeparn (45 min.). The peak concentration of diazepam was greater when given intranasally (162 ng/ml) than rectally (128 ng/ml).

Enhanced Permeation of Diazepam through Madin-Darby Canine Kidney (MDCK) II Epithelial Cell Monolayer from Supersaturated Solutions

Overview

In the studies above, it was shown that diazepam (DZP) can be formulated in a supersaturated state in water/glycofurol(GF) cosolvent systems. The temporal stability of these solutions depended on degree of supersaturation (DS), and many formulations were stable for >20 min, long enough to permit absorption of DZP across the nasal mucosa before the onset of crystallization/precipitation of DZP. It was also shown that up to DS=3, flux rate of DZP across model silicone membranes was proportional to DZP concentration.

In the following studies, permeation of DZP across a confluent cell culture model was examined. In addition, effects of cosolvents on cell culture viability over the times of exposure were assessed.

Materials and methods

Cell Culture

Maden Darby Canine Kidney II wild type (MDCKII-WT) cells were seeded at 43,000 cells/cm² on six-well polyester membrane inserts (Transwell®) with a pore size of 0.4 μm. Cells were maintained in Dulbecco's modified Eagle's medium supplemented with 10% fetal bovine serum and 1% penicillin and streptomycin at 37° C. in a humidified incubator with 5% CO₂. Media were changed every other day, and cell monolayers were cultured for 4 days before use.

Cell Viability

Viability of MDCKII-WT cells was measured by incubating the cells in suspension for 30, 60, and 120 min in various GF/buffer compositions at 37° C. Trypan blue dye was then added into the cell suspension, and incubated for 10 min. A sample was drawn from the suspension and spread on a hemocytometer, and counts of live (transparent) and dead (blue) cells were made.

Trans-Epidermal Electrical Resistance (TEER) Measurement

The electrical resistance of each monolayer was measured at three positions using an EVOM™ epithelial volt-ohm-meter. The background resistance was subtracted from each monolayer resistance value. Monolayer resistance values were multiplied by the membrane area (4.67 cm²) and averaged to calculate TEER values for each monolayer.

Transport Studies

Upon cell confluence, cell monolayers were washed twice and preincubated with assay buffer (containing 122 mM NaCl, 25 mM NaHCO₃, 10 mM glucose, 10 mM HEPES, 3 mM KCl, 1.2 mM MgSO₄, 1.4 mM CaCl₂, and 0.4 mM K₂HPO₄, pH 7.4) at 37° C. for 30 min. Thereafter, 1.5 ml of drug ([¹⁴C]mannitol or DZP)-in-glycofurol/assay buffer (GF/AB) solution and 2.6 ml of drug-free GF/AB solution were introduced into the apical (donor) and basal (receptor) side, respectively. Monolayers were incubated at 37° C. while rotating on an orbital shaker at 60 rpm. Samples were taken from the receptor side every 5 min for 30 min.

Permeability of [¹⁴C]mannitol was measured to monitor the integrity of tight junction under different GF/AB cosolvent mixtures with varying GF content from 0 to 50 vol %. The percentage mass transported of mannitol from the starting amount was calculated from the dpm measured in a Beckman LS 6000SE liquid scintillation counter and plotted as a function of time, the slope of which was used to calculate the permeability of mannitol according to the following equation:
Permeability=(slope·V)/(100·A)

where V and A are the volume of the donor chamber and the membrane surface area, respectively.

Permeation of DZP across the cell monolayer was measured from solutions at different degrees of saturation (S) in a 30/70 GF/AB vehicle. The cumulative amount of permeated DZP through the cell monolayer was measured by HPLC and plotted as a function of time. Steady state flux was determined by linear regression on these plots.

Results

Effect of GF on MDCK Cell Viability

FIG. 16 presents the composition-dependent viability of MDCK cells treated with GF for 30 min, 1 hour and 2 hours. The results show that GF had cytotoxic effects in a composition and time-dependent manner. A 30 min-treatment with GF up to 50 vol % induced insignificant toxicity towards MDCK cells, more than 90% cells were viable. A 1 h exposure to GF led to a marked decease in cell viability at GF compositions above 30%. A 2 h exposure resulted in 100% lethality at GF compositions equal to or more than 20%. Since the interest was only in studying DZP permeation through MDCK cell monolayers in a time period of 30 mins, these cell viability results were acceptable.

Effect of GF on MDCK Cell Monolayer Integrity

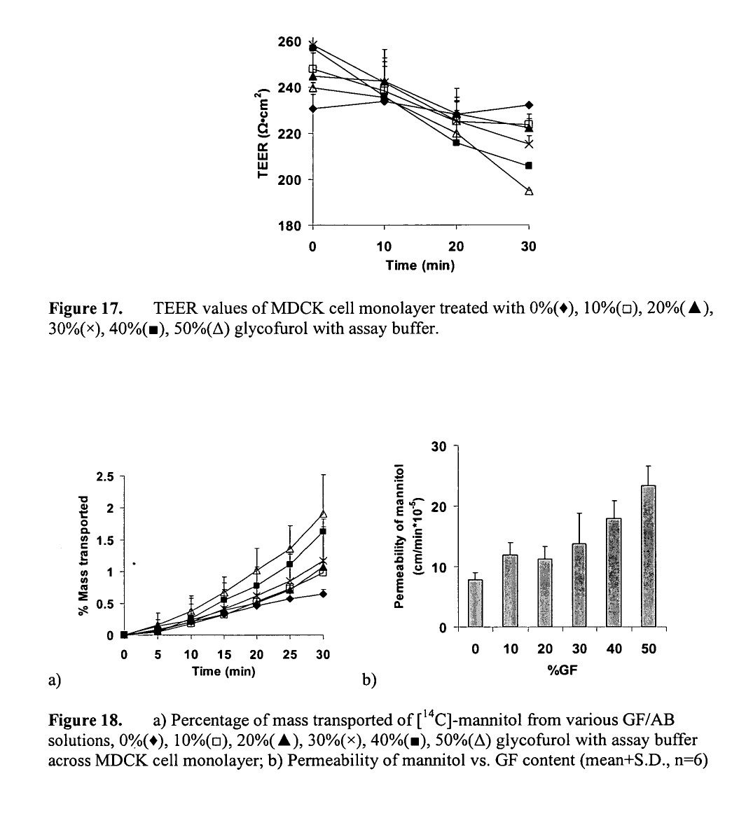

MDCKII-WT cell monolayer integrity under various GF/AB cosolvent systems was monitored by measuring TEER values and the permeability of [¹⁴C]mannitol. As shown in FIG. 17, TEER values decrease slightly over 30 min, this effect becoming more pronounced with increasing GF composition. However, TEER values never approach 100 Ω·cm², the threshold value below which MDCKII monolayers are generally considered to be “leaky”.

Barrier function of MDCKII monolayer was then assessed more thoroughly by examining mannitol permeability. As shown in FIG. 18, with increasing GF content in the cosolvent mixtures, permeability of mannitol increases. Thus it was considered that induction of increased mannitol permeability correlates with lowered TEER. Note that for all GF/AB cosolvent systems, at t=30 min, the percentage mass transported of mannitol is well below 5%, the upper limit for a cell monolayer with good barrier function.

Effect of DZP on MDCK Cell Monolayer Integrity

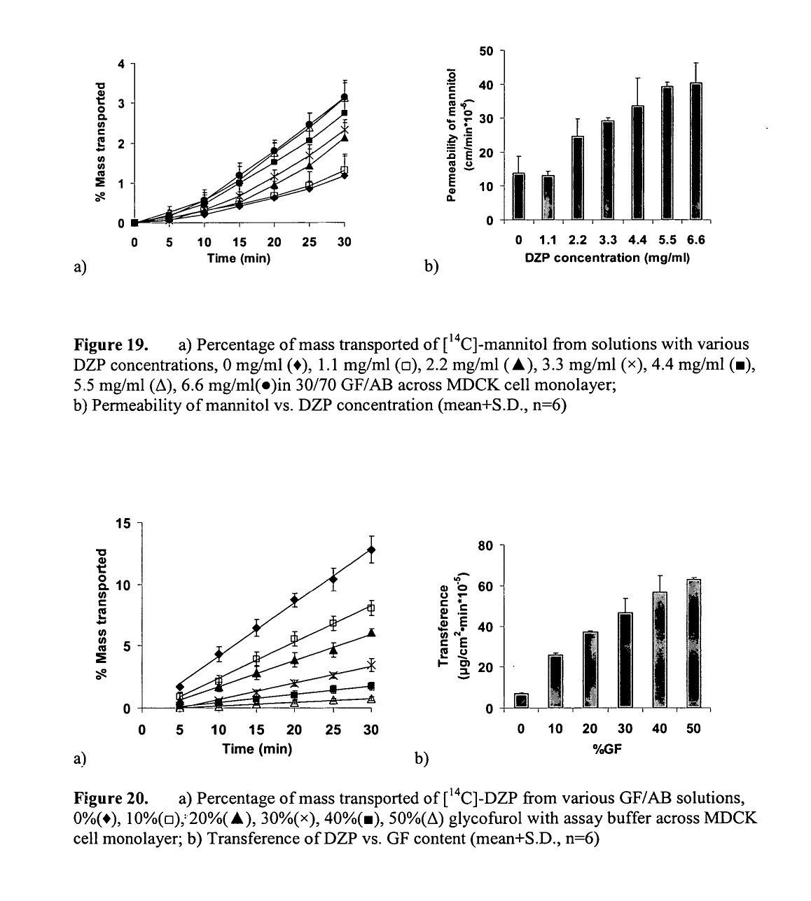

In order to monitor the effect of DZP on monolayer integrity, mannitol permeability in the presence of increasing concentrations of DZP in a 30/70 GF/assay buffer cosolvent mixtures was determined. Results are shown in FIG. 19. Exposure of the monolayers to DZP induced an increase of mannitol permeability in a concentration-dependent manner. At DZP concentration of 6.6 mg/ml and at t=30 min, the percentage mass transported of mannitol is still below 5%, the upper limit for a cell monolayer with effective barrier function.

Effect of GF on MDCK Cell Monolayer Transference

After the viability and integrity tests, the effect of GF on MDCK cell monolayer's transference was evaluated by measuring permeation of DZP from upstream solutions with various GF compositions from 0 to 50 vol %. Transference of DZP through MDCK cell monolayer is plotted as a function of GF composition in FIG. 20. It was determined that transference increased with increasing GF content in the upstream vehicle, indicating that GF has effect on MDCK cell monolayer. On the other hand, GF might be served as a permeation enhancer for DZP penetration through MDCK cell monolayers.

Effect of DZP on MDCK Cell Monolayer Permeability

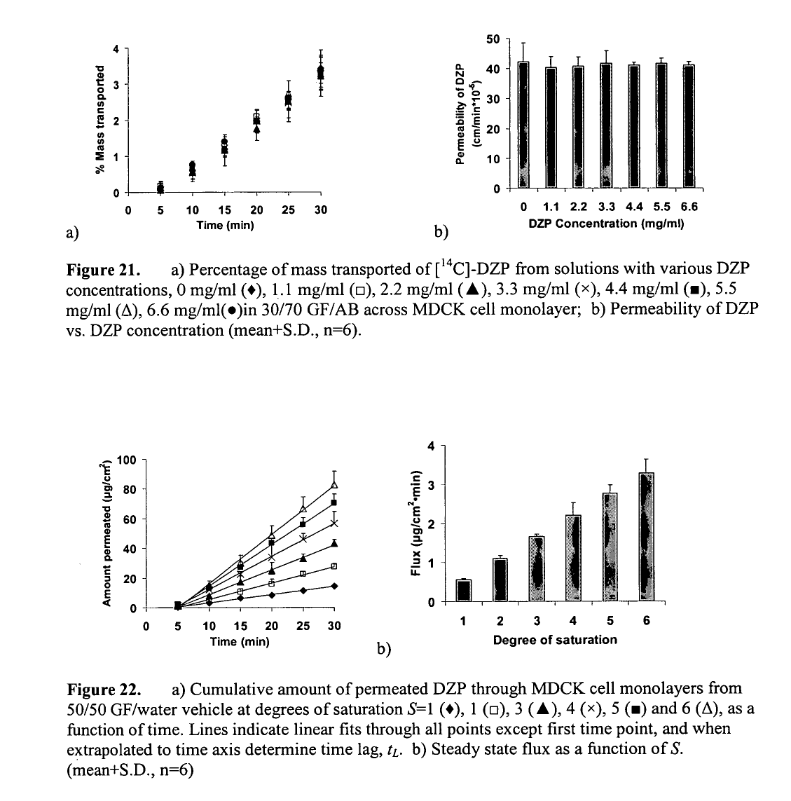

In addition to GF, DZP itself might also have effect on monolayer's permeability. To check against this possibility, permeation of radio-labeled DZP was measured from upstream solutions of cold DZP at different concentrations in a 30/70 GF/assay buffer vehicle, and with hot DZP at the same concentration. The results are shown in FIG. 21. Essentially identical permeability was obtained from solutions with different DZP concentrations, demonstrating that at the concentrations and exposure times studied, DZP has no effect on MDCK cell monolayer permeability.

Effect of supersaturation on DZP Permeation through MDCK Cell Monolayer

The cumulative permeation of DZP across MDCKII-WT monolayers from solutions with degrees of saturation S from 1 to 6 in a 30/70 GF/AB vehicle is plotted as a function of time in FIG. 22a, and the steady state fluxes calculated from these data are plotted against S in FIG. 22b. Steady state flux increased linearly with increasing degree of saturation, demonstrating the ability to enhance transport using supersaturation.

DZP Permeation Through Blank Filter

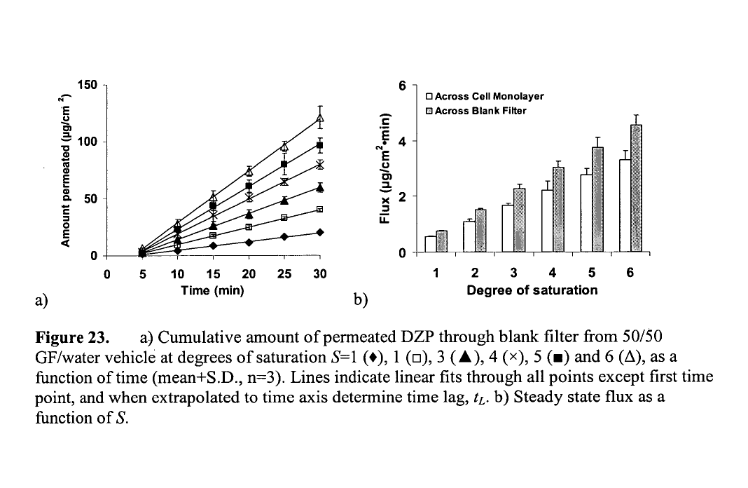

The cumulative permeation of DZP across blank filters without growing with MDCK cell monolayers from solutions with degrees of saturation S from 1 to 6 in a 30/70 GF/AB vehicle is plotted as a function of time in FIG. 23a, and the steady state fluxes calculated from these data are plotted against S in FIG. 23b. Steady state flux increased linearly with increasing degree of saturation, demonstrating the ability to enhance transport using supersaturation. In addition, compared with DZP fluxes through MDCK cell monolayers, DZP fluxes through blank filters are greater.

### CONCLUSION

Permeation of diazepam across Madin-Darby Canine Kidney (MDCK) cell monolayer, chosen as an in vitro model for nasal mucosa, was shown was enhanced with supersaturated solutions, demonstrating the ability to increase the rate of diazepam absorption using supersaturation. MDCK cell monolayer's permeability was affected by glycofurol, but not by diazepam itself. A 30 min-exposure with glycofurol up to 50 vol % induced insignificant toxicity towards MDCK cells, more than 90% cells are viable. Cell monolayer integrity was monitored by measuring transepithelial electrical resistance (TEER) and permeability of [¹⁴C]mannitol. The results indicate that MDCK cell monolayers exhibited good barrier function under experimental conditions.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claim.

I claim:

1. A composition comprising:
a supersaturated solution of a benzodiazepine, water and glycofurol.

2. The composition of claim 1, wherein the benzodiazepine is diazepam.

3. The composition of claim 2, wherein the concentration of diazepam is between about 10 mg/ml and about 60 mg/ml.

4. The composition of claim 1, wherein the concentration of the benzodiazepine is about 40 mg/ml.

5. The composition of claim 4, wherein the benzodiazepine is diazepam.

6. The composition of claim 1, wherein the glycofurol has the structure 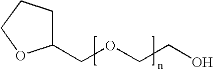
wherein n is 0 to 5.

7. The composition of claim 1, wherein the glycofurol percentage of the water and glycofurol combination is between about 40 percent and about 65 percent.

8. The composition of claim 7, wherein the glycofurol percentage of the water and glycofurol combination is between about 45 percent and about 60 percent.

9. The composition of claim 7, wherein the benzodiazepine concentration is about 40 mg/ml.

10. The composition of claim 8, wherein the benzodiazepine concentration is about 40 mg/ml.

11. The composition of claim 9, wherein the benzodiazepine is diazepam.

12. The composition of claim 10, wherein the benzodiazepine is diazepam.

13. A method for intranasal administration of a benzodiazepine, comprising the step of:
providing a therapeutically effective amount of a supersaturated benzodiazepine solution via intranasal administration, wherein said supersaturated solution comprises:
a benzodiazepine, water and a glycofurol.

14. The method of claim 13, wherein the benzodiazepine is diazepam.

15. The method of claim 14, wherein the concentration of diazepam is between about 10 mg/ml and about 60 mg/ml.

16. The method of claim 13, wherein the concentration of the benzodiazepine is about 40 mg/ml.

17. The method of claim 16, wherein the benzodiazepine is diazepam.

18. The method of claim 13, wherein the glycofurol has the structure 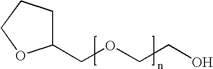
wherein n is 0 to 5.

19. The method of claim 13, wherein the glycofurol percentage of the water and glycofurol combination is between about 40 percent and about 65 percent.

20. The method of claim 19, wherein the glycofurol percentage of the water and glycofurol combination is between about 45 percent and about 60 percent.

21. The method of claim 19, wherein the benzodiazepine concentration is about 40 mg/ml.

22. The method of claim 20, wherein the benzodiazepine concentration is about 40 mg/ml.

23. The method of claim 21, wherein the benzodiazepine is diazepam.

24. The method of claim 22, wherein the benzodiazepine is diazepam.

25. The method of claim 13, wherein the supersaturated benzodiazepine solution is administered by spray.

26. The method of claim 13, wherein the supersaturated benzodiazepine solution is administered by drops.

27. The method of claim 25, wherein the spray is created via chaotic advection mixing in a microfluidic delivery chamber, or by turbulent mixing.

28. A method to sedate a mammal comprising the nasal administration of a composition comprising a therapeutically effective amount of a supersaturated solution of a benzodiazepine, water and a glycofurol.

29. The method of claim 28, wherein the benzodiazepine is diazepam.

30. The method of claim 29, wherein the concentration of diazepam is between about 10 mg/ml and about 60 mg/ml.

31. The method of claim 28, wherein the concentration of the benzodiazepine is about 40 mg/ml.

32. The method of claim 31, wherein the benzodiazepine is diazepam.

33. The method of claim 28, wherein the glycofurol has the structure 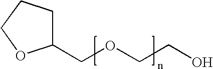
wherein n is 0 to 5.

34. The method of claim 28, wherein the glycofurol percentage of the water and glycofurol combination is between about 40 percent and about 65 percent.

35. The method of claim 34, wherein the glycofurol percentage of the water and glycofurol combination is between about 45 percent and about 60 percent.

36. The method of claim 34, wherein the benzodiazepine concentration is about 40 mg/ml.

37. The method of claim 35, wherein the benzodiazepine concentration is about 40 mg/ml.

38. The method of claim 36, wherein the benzodiazepine is diazepam.

39. The method of claim 37, wherein the benzodiazepine is diazepam.

40. The method of claim 28, wherein the supersaturated benzodiazepine solution is administered by spray.

41. The method fo claim 28, wherein the supersaturated benzodiazepine solution is administered by drops.

42. The method of claim 28, wherein the spray is created via chaotic mixing in a microfluidic delivery chamber.

43. A method to treat epilepsy comprising the nasal administration of a composition comprising a therapeutically effective amount of a supersaturated solution of a benzodiazepine, water and a glycofurol.

44. The method of claim 43, wherein the benzodiazepine is diazepam.

45. The method of claim 44, wherein the concentration of diazepam is between about 10 mg/ml and about 60 mg/ml.

46. The method of claim 43, wherein the concentration of the benzodiazepine is about 40 mg/ml.

47. The method of claim 46, wherein the benzodiazepine is diazepam.

48. The method of claim 43, wherein the glycofurol has the structure 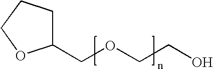
wherein n is 0 to 5.

49. The method of claim 43, wherein the glycofurol percentage of the water and glycofurol combination is between about 40 percent and about 65 percent.

50. The method of claim 49, wherein the glycofurol percentage of the water and glycofurol combination is between about 45 percent and about 60 percent.

51. The method of claim 49, wherein the benzodiazepine concentration is about 40 mg/ml.

52. The method of claim 50, wherein the benzodiazepine concentration is about 40 mg/ml.

53. The method of claim 51, wherein the benzodiazepine is diazepam.

54. The method of claim 52, wherein the benzodiazepine is diazepam.

55. The method of claim 53, wherein the supersaturated benzodiazepine solution is administered by spray.

56. The method fo claim 53, wherein the supersaturated benzodiazepine solution is administered by drops.

57. The method of claim 55, wherein the spray is created via chaotic advection mixing in a microfluidic delivery chamber, or by turbulent mixing.
